(19) United States
(12) Patent Application Publication
(10) Pub. No.: US 2022/0075168 A1
(43) Pub. Date: Mar. 10, 2022

(54) MOUNTABLE DENTAL/SURGICAL MICROSCOPE

(71) Applicant: Ho Jun Eom, Burnie, Tasmania (AU)

(72) Inventor: Ho Jun Eom, Burnie, Tasmania (AU)

(21) Appl. No.: 17/417,881

(22) PCT Filed: Jan. 6, 2020
(86) PCT No.: PCT/IB2020/050066

§ 371 Date: Jun. 24, 2021

Related U.S. Application Data
(60) Provisional application No. 62/932,341, filed on Nov. 7, 2019.
(60) Provisional application No. 62/788,240, filed on Jan. 4, 2019.

(51) Int. Cl. G02B 21/00; A61B 90/25; A61B 1/24; G02B 21/36; G02B 21/02; G02B 21/22; G02B 21/08

(52) U.S. Cl. CPC G02B 21/0012; A61B 90/25; A61B 1/24; G02B 21/08; G02B 21/025; G02B 21/22; G02B 21/361

(57) ABSTRACT

A mountable dental/surgical microscope is an apparatus that attaches to a medical overhead light. The apparatus includes an overhead attachment mechanism, a positioning arm, a stereoscopic ocular, and a light-modifying scope. The overhead attachment mechanism attaches the apparatus to the medical overhead light. The positioning arm allows a user to reposition the apparatus in order to better view an object through the apparatus. The light-modifying scope condenses, collimates, focuses, and magnifies the light traversing through the apparatus, which allows the user to better view the object through the apparatus. The stereoscopic ocular allows the user to comfortably view the object by redirecting an optical image of the object to a desirable position offset from the light-modifying scope.

### CROSS-REFERENCE TO RELATED APPLICATIONS

This application is a national stage application of PCT/IB2020/050066. This application claims priorities from PCT Application No. PCT/IB2020/050066, filed Jan. 6, 2020, and from Provisional Application No. 62/932,341, filed Jun. 13, 2019, and Provisional Application No. 62/788,240, filed Jan. 4, 2019, the content of which are incorporated herein in the entirety by references.

### FIELD OF THE INVENTION

The present invention generally relates to medical systems and methods. More specifically, the present invention relates to medical microscopes which can be attached to secondary objects such as light fixtures.

### BACKGROUND OF THE INVENTION

Currently, most medical microscopes on the market require a heavy stand or a ceiling mount to be installed within a medical facility. The current medical microscopes stands/mount can be provided as separate structures or can be integrated to existing structures. Unfortunately, the current medical microscope stands/mounts can often be bulky and can be difficult to install and/or remove. Further, the currently available medical microscopes stands/mounts can sometimes be obstructive to the dental staff and not practical to use while performing various medical procedures on patients. In addition, current medical microscopes often provide their own illumination system, requiring a power source to be connected to the medical microscopes and adding weight to the overall structure of the medical microscope.

An objective of the present invention is to provide a lightweight mountable dental/surgical microscope which can be attached to pre-existing dental fixtures or similar medical light fixtures. In the preferred embodiment of the present invention, the mountable dental/surgical microscope does not comprise an integrated illumination system. Instead, the mountable dental/surgical microscope utilizes the illumination provided by pre-existing light fixtures to which the mountable dental/surgical microscope is attached. The mountable dental/surgical microscope is easy to install and/or remove and can fit in a small storage space such as within a suitcase for easy transportability. The mountable dental/surgical microscope comprises an objective pointed towards the mouth of the patient and an eyepiece which allows users to look through. Further, the mountable dental/surgical microscope comprises a prism system which creates an angle between the objective and eyepiece preferably between 20 degrees and 90 degrees. The inter-pupillary distance of the mountable dental/surgical microscope is also adjustable as well as other optical variables.

### SUMMARY OF THE INVENTION

The present invention provides a mountable dental/surgical microscope which can be attached to a pre-existing dental overhead light or similar medical light fixture, such as veterinarian surgical overhead lights, military/humanitarian surgical field overhead lights, etc. The mountable dental/surgical microscope will focus the light provided by the light fixture to be more concentrated onto the desired area. Further, the mountable dental/surgical microscope will magnify and visualize biological tissue, primarily teeth and surrounding gingiva. In the preferred embodiment of the present invention, the mountable dental/surgical microscope comprises an attaching mechanism which allows the mountable dental/surgical microscope to be attached to a light fixture. The mountable dental/surgical microscope further comprises an adjustment mechanism which allows the adjustment of the microscope light collimating/focusing device relative to the overhead light source. Additionally, the adjustment mechanism of the mountable dental/surgical microscope allows the adjustment of the position and orientation of the mountable dental/surgical microscope relative to the light fixture. Furthermore, the mountable dental/surgical microscope comprises a plurality of accessories which can be manually attached/detached to the overhead light to adjust the stiffness of structure of the overhead light.

### BRIEF DESCRIPTION OF THE DRAWINGS

FIG. 1 is a right-front-top perspective view of the present invention.

FIG. 2 is a right-front-rear perspective view of the present invention.

FIG. 3 is a left-front-bottom perspective view of the present invention.

FIG. 4 is a right-side view of the present invention.

FIG. 5 is an internal schematic view of the present invention.

### DETAIL DESCRIPTIONS OF THE INVENTION

All illustrations of the drawings are for the purpose of describing selected versions of the present invention and are not intended to limit the scope of the present invention. The present invention provides a mountable dental/surgical microscope which can be attached to a pre-existing overhead light fixture such as a dental overhead light or similar medical light fixtures. More specifically, a preferred embodiment of the present invention is able to condense and re-center a plurality of multi-directional light rays from the pre-existing overhead light fixture into a plurality of parallel light rays to improve the illumination conditions of an object, wherein the object can be a surgical procedure, a specimen, a dental procedure, or any other types of precisely conducted procedures.

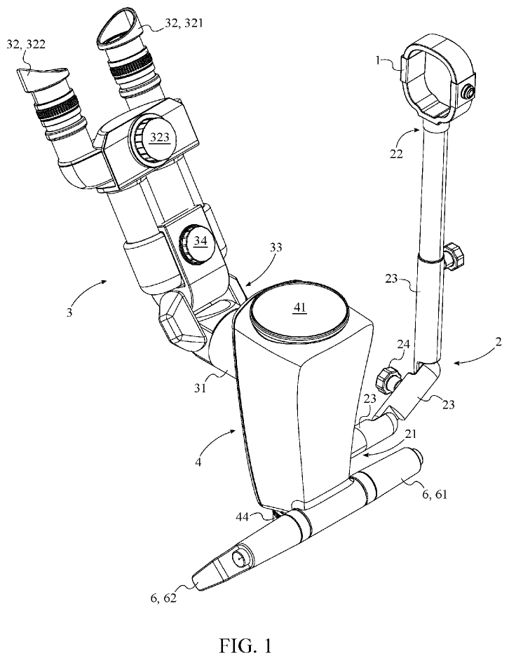

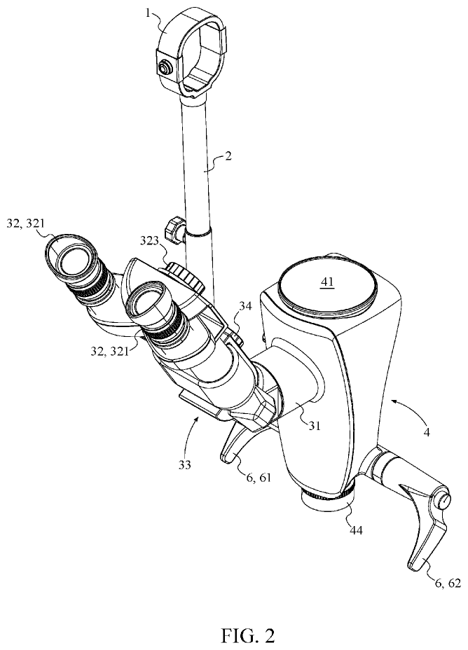

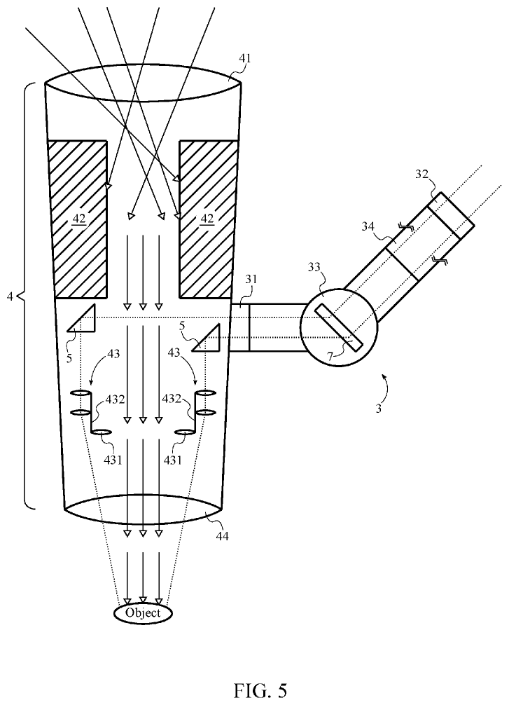

The preferred embodiment of the present invention comprises an overhead attachment mechanism 1, a positioning arm 2, a stereoscopic ocular 3, and a light modifying scope 4 as shown in FIGS. 1 and 2. The overhead attachment mechanism 1 is a fastening bracket and allows the present invention to be easily attached or detached to and from the pre-existing overhead light fixture. The positioning arm 2 functions as the supporting structural body between the overhead attachment mechanism 1 and the light modifying scope 4. The stereoscopic ocular 3 allows both of the user's eyes to receive the optical image of the object that has been generated and treated by the present invention. As shown in FIGS. 1 and 2, the stereoscopic ocular 3 comprises an optical inlet 31 and an optical outlet 32. The light-modifying scope 4 is the critical functional component of the present invention as the plurality of multi-directional light rays from the pre-existing overhead light fixture is condensed and re-centered into the plurality of parallel light rays through the light-modifying scope 4. As shown in FIG. 5, the light-modifying scope 4 comprises a collector lens 41, a light-collimating mechanism 42, a light-refracting mechanism 43, and an objective lens 44.

In reference to the general configuration of the present invention, the stereoscopic ocular 3 and a proximal arm end 21 of the positioning arm 2 are laterally mounted to the light-modifying scope 4. The stereoscopic ocular 3 and the proximal arm end 21 are positioned offset from each other about the light-modifying scope 4 so that the positioning arm 2 and the stereoscopic ocular 3 are able to independently operate on the present invention. The overhead attachment mechanism 1 is mounted onto a distal arm end 22 of the positioning arm 2 so that the light-modifying scope 4 can be positioned away from the pre-existing overhead light fixture. The collector lens 41 is in optical communication with the objective lens 44 through the light-collimating mechanism 42 within the light-modifying scope 4. More specifically, the collector lens 41 is oriented towards the pre-existing overhead light fixture and receives the plurality of multi directional light rays. Then, the collector lens 41 is able to collect the plurality of multi directional light rays into the light-collimating mechanism 42. The objective lens 44 is oriented towards the object and emits the plurality of parallel light rays as the light-collimating mechanism 42 aligns and condenses the plurality of multi-directional light rays into the plurality of parallel light rays. Furthermore, the optical inlet 31 is in optical communication with the optical outlet 32 so that the user is able to view the object. More specifically, the optical inlet 31 is positioned adjacent to the light-modifying scope 4 so that an optical image of the object can be displayed through the optical outlet 32.

Furthermore, the optical outlet 32 is positioned offset from the light-modifying scope 4 thus providing sufficient distance between the user and the light-modifying scope 4.

As can be seen in FIG. 5, the light-refracting mechanism 43 that is used to magnify the optical image may comprise at least one zoom lens 431 and at least one turret 432 (i.e. only if the light-refracting mechanism 43 can actuate two or more zoom lenses will the light-refracting mechanism 43 need to comprise a turret). The objective lens 44 is in optical communication the optical inlet 31 through the at least one zoom lens 431. The at least one zoom lens 431 is operatively mounted to the light-modifying scope 4 by the at least one turret 432. More specifically, the at least one turret 432 is used to selectively magnify the optical image traversing from the objective lens 44, through the at least one zoom lens 431, and into the optical inlet 31 of the stereoscopic ocular 3. In other words, multiple zoom lenses are attached to the at least one turret 432 that rotates in and out of an optical path, and the at least one zoom lens 431 mounted through the at least one turret 432 has different curvatures to change the zoom.

The present invention further comprises an optical redirection system 5 as shown in FIG. 5. The optical redirection system 5 is mounted within the light-modifying scope 4, and the light-refracting mechanism 4 is in optical communication with the optical inlet 31 through the optical redirection system 5. The optical redirection system 5 is used to transversely reorients a plurality of received light rays away from an optical path between the collector lens 41 and the objective lens 44, wherein the plurality of received light rays have reflected off the object and traversed through the objective lens 44. In other words, the optical redirection system 5 allows the optical image of the object to be precisely redirected into the optical inlet 31 thus maximizing the clarity of the object while the user is able to comfortably view the optical image. Depending upon different embodiments of the present invention, the optical redirection system 5 can be a cubic beam splitter, a prism, a mirror, or any other combination of optical elements.

The present invention further comprises a handle as shown in FIG. 1. The at least one handle 6 is laterally connected to the light-modifying scope 4 so that the user can easily maneuver the positioning of the present invention with respect to the exact location of the object. Preferably, the at least one handle 6 is laterally connected to a housing of the light-modifying scope 4 so that the at least one handle 6 can be oriented outward. Furthermore, the at least one handle 6 is ergonomically shaped to reduce fatigue and discomfort of the user's hand. Optionally, the exact orientation of the at least one handle 6 can be either rotated or changed about the light-modifying scope 4 so that the user is able to adjust the at least one handle 6 upon user's preference.

In the preferred embodiment of the present invention, the at least one handle 6 comprises a first handle 61 and a second handle 62 as shown in FIG. 1. More specifically, the first handle 61 and the second handle 62 are positioned opposite to each other about the light-modifying scope 4 thus allowing the user to grasp the present invention with their left hand, right hand, or both hands. The first handle 61 and the second handle 62 are also positioned adjacent to the objective lens 44 thus minimizing the distance between the object and the light-modifying scope 4. As a result, the user is able to easily adjust small angular positioning of the light-modifying scope 4 with precise changes or movements.

The optical outlet 32 that displays the object to the user preferably comprises a first eyepiece 321, a second eyepiece 322, and a spacing-adjustment mechanism 323. The first eyepiece 321 and the second eyepiece 322 are positioned offset from each other so that the user is able to align and rest their left and right eyes on the optical outlet 32. As a result, the first eyepiece 321 and the second eyepiece 322 are able to function as an optical platform that view the optical image of the object through the optical image. The first eyepiece 321 and the second eyepiece 322 are operatively coupled to each other by the spacing-adjustment mechanism 323, wherein the spacing-adjustment mechanism 323 is used to adjust an interpupillary distance between the first eyepiece 321 and the second eyepiece 322. In other words, the spacing-adjustment mechanism 323 adjusts the linear space between the first eyepiece 321 and the second eyepiece 322 thus allowing the optical image to completely fill the field of view of the first eyepiece 321 and the second eyepiece 322, while minimizing the amount of extraneous light.

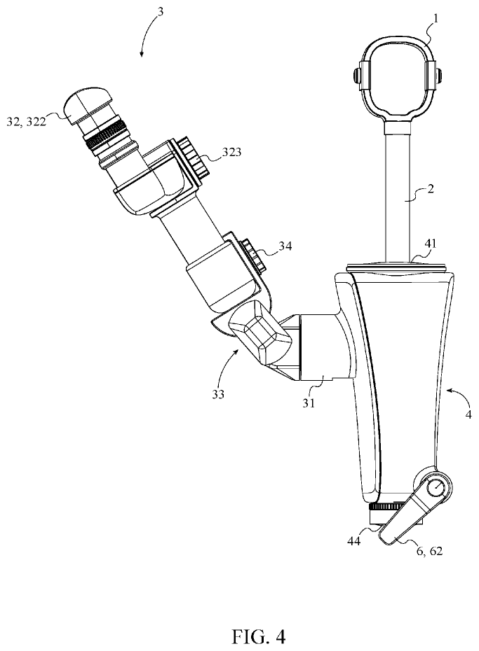

In order to provide the user with better clarity on an optical image of the object, the spectroscopic ocular 3 may further comprise a light-focusing mechanism 34, which is shown in FIGS. 1 through 5. The light-focusing mechanism 34 is preferably positioned in between the optical inlet 31 and the optical outlet 32 so that the optical inlet 31 can be in optical communication with the optical outlet 32 through the light-focusing mechanism 34. This arrangement allows the light-focusing mechanism 34 to either lengthen or shorten the optical working distance between the optical inlet 31 and the optical outlet 32 and to consequently adjust the focal length for the overall optical system of the present invention.

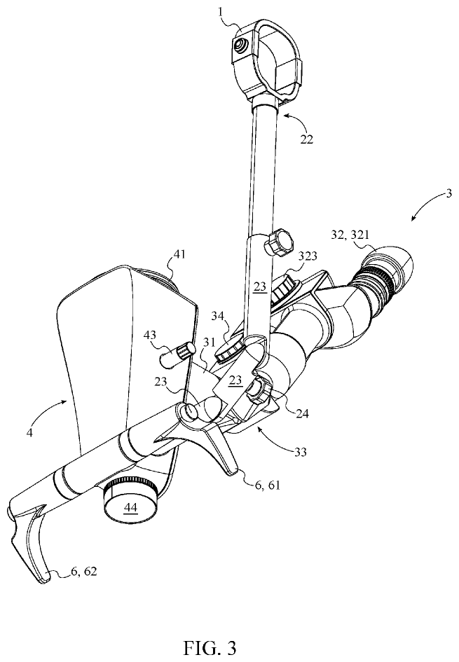

The positioning arm 2 that defines the distance between the overhead attachment mechanism 1 and the light-modifying scope 4 comprises a series of arm members 23 and a plurality of movement dampeners 24 as shown in FIGS. 1 and 3. The series of arm members 23 is a plurality of elongated structural bodies that enables multi-directional movement of the light-modifying scope 4. Thus, the user can lock or unlock the relative positioning between the light-modifying scope 4 and the overhead light. The plurality of movement dampeners 24 is used to control, resist, and/or lock members so that the series of arm members 23 is able to stabilize a defined positioning of the light-modifying scope 4. For example, each of the plurality of movement dampeners 24 can be, but is not limited to, a pneumatic cylinder and piston assembly, a simple locking nut, a ball-and-socket joint that can be tightened, or any other mechanical system for the purpose of movement restriction and/or dampening. In reference to the general configuration of the positioning arm 2, each pair of adjacent members from the series of arm members 23 is pivotably and terminally connected to each other. Furthermore, each pair of adjacent members from the series of arm members 23 is laterally connected to each other by a corresponding dampener from the plurality of movement dampeners 24 thus providing individual stabilization with respect to each pair of adjacent members.

The present invention further comprises at least one optical swivel system 7 that eases the viewing process of the optical image through the first eyepiece 321 and the second eyepiece 322 for the user without moving around too much. In order to incorporate the at least one optical swivel system 7, the stereoscopic ocular 3 further comprises at least one movement joint 33. In reference to FIGS. 1 through 5, the at least one movement joint 33 is positioned in between the optical inlet 31 and the optical outlet 32 so that the at least one optical swivel system 7 can be operatively integrated into the at least one movement joint 33, wherein the at least one optical swivel system 7 is used to maintain optical communication between the optical inlet 31 and the optical outlet 32 in both a linear alignment and a non-linear alignment. The at least one optical swivel system 7 can be any combination of prisms, lenses, mirrors, and/or other optical elements that is used to guide the optical image through the at least one movement joint 33 without optically distorting the optical image.

Although the invention has been explained in relation to its preferred embodiment, it is to be understood that many other possible modifications and variations can be made without departing from the spirit and scope of the invention as hereinafter claimed.

What is claimed is:

1. A mountable dental/surgical microscope comprises:
an overhead attachment mechanism;
a positioning arm;
a stereoscopic ocular;
a light-modifying scope;
the positioning arm comprises a proximal arm end and a distal arm end; the light-modifying scope comprises a collector lens, a light-collimating mechanism, a light-refracting mechanism, and an objective lens;
the stereoscopic ocular comprises an optical inlet and an optical outlet; the stereoscopic ocular and the proximal arm end being laterally mounted to the light-modifying scope;
the stereoscopic ocular and the proximal arm end being positioned offset from each other about the light-modifying scope;
the overhead attachment mechanism being mounted onto the distal arm end;
the collector lens being in optical communication with the objective lens through the light-collimating mechanism;
the objective lens being in optical communication the optical inlet through the light-refracting mechanism;
the optical inlet being positioned adjacent to the light-modifying scope; the optical outlet being positioned offset from the light-modifying scope; and
the optical inlet being in optical communication with the optical outlet.

2. The mountable dental/surgical microscope as claimed in claim 1 comprises:
an optical redirection system;
the optical redirection system being mounted within the light-modifying scope; and
the light-refracting mechanism being in optical communication with the optical inlet through the optical redirection system.

3. The mountable dental/surgical microscope as claimed in claim 1 comprises:
at least one handle; and the at least one handle being laterally connected to the light-modifying scope.

4. The mountable dental/surgical microscope as claimed in claim 3 comprises:
the at least one handle comprises a first handle and a second handle;
the first handle and the second handle being positioned opposite to each other about the light-modifying scope; and
the first handle and the second handle being positioned adjacent to the objective lens.

5. The mountable dental/surgical microscope as claimed in claim 1 comprises:
the optical outlet comprises a first eyepiece, a second eyepiece, and a spacing-adjustment mechanism;
the first eyepiece and the second eyepiece being positioned offset from each other; and
the first eyepiece and the second eyepiece being operatively coupled to each other by the spacing-adjustment mechanism, wherein the spacing-adjustment mechanism is used to adjust an interpupillary distance between the first eyepiece and the second eyepiece.

6. The mountable dental/surgical microscope as claimed in claim 1 comprises:
the light-refracting mechanism comprises at least one zoom lens and at least one turret;
the objective lens being in optical communication the optical inlet through the at least one zoom lens; and the at least one zoom lens being operatively mounted within the light modifying scope by the at least one turret, wherein the at least one turret is used to selectively focus and magnify an optical image traversing from the objective lens to the stereoscopic ocular with the at least one zoom lens.

7. The mountable dental/surgical microscope as claimed in claim 1 comprises:
the positioning arm comprises a series of arm members and a plurality of movement dampeners;
each pair of adjacent members from the series of arm members being pivotably and terminally connected to each other; and
each pair of adjacent members from the series of arm members being laterally connected to each other by a corresponding dampener from the plurality of movement dampeners.

8. The mountable dental/surgical microscope as claimed in claim 1 comprises:
at least one optical swivel system;
the stereoscopic ocular further comprises at least one movement joint; the at least one movement joint being positioned in between the optical inlet and the optical outlet; and the at least one optical swivel system being operatively integrated into the at least one movement joint, wherein the at least one optical swivel system is used to maintain optical communication between the optical inlet and the optical outlet in both a linear alignment and a non-linear alignment.

9. The mountable dental/surgical microscope as claimed in claim 1 comprises:
the stereoscopic ocular further comprises a light-focusing mechanism; the light-focusing mechanism being positioned in between the optical inlet and the optical outlet; and the optical inlet being in optical communication with the optical outlet through the light-focusing mechanism.
